(19) United States
(12) Patent Application Publication
(10) Pub. No.: US 2011/0282461 A1
(43) Pub. Date: Nov. 17, 2011

(54) STENT

(76) Inventors: Kyong-Min SHIN, Seoul (KR); Byung-Cheol Myung, Kyunggi-do (KR)

(21) Appl. No.: 13/095,370

(22) Filed: Apr. 27, 2011

(30) Foreign Application Priority Data
May 14, 2010 (KR) 10-2010-0045340

(51) Int. Cl. A61F 2/04

(52) U.S. Cl. 623/23.7

(57) ABSTRACT

Disclosed herein is a stent implanted in the body of a patient. The stent includes a hollow cylindrical stent body which is made of a super-elastic shape memory alloy and has an expanded diameter part on one end thereof, and a support stent which has an elastic spherical structure and is fitted over the cylindrical stent body behind the expanded diameter part. The stent is implanted in the body such that food that has passed through the stomach is prevented from mixing with bile or pancreatic juice in the duodenum and moves directly into the small intestine to prevent the duodenum from absorbing nutrients of the food while the small intestine directly digests the food and absorbs the nutrients, thus minimizing a nutrient absorption rate, thereby preventing the obesity of the patient.

### CROSS-REFERENCE TO RELATED APPLICATIONS

The present application claims priority of Korean patent application number 10-2010-0045340 filed on May 14, 2010, which is incorporated herein by reference in its entirety.

### BACKGROUND OF THE INVENTION

1. Field of the Invention

The present invention relates generally to a stent which is implanted in the stomach of a patient who has undergone a gastrectomy so that when the patient takes in food, the stent prevents an inflammation from occurring on the suture region of the stomach that would result in contact being made with the food, and prevents the suture region from being reopened by an inflammation or a ligation of the suture region being damaged, thus reducing the time required for the suture region to heal and, more particularly, to a stent which is configured such that food that has passed through the stomach is prevented from mixing with bile or pancreatic juice in the duodenum and is directly moved into the small intestine to prevent the duodenum from absorbing the nutrients of the food while the small intestine directly digests the food and absorbs the nutrients, thus minimizing a nutrient absorption rate, thereby preventing the obesity of the patient.

2. Description of the Related Art

Generally, BMI (body mass index, kg/m²) is defined as the individual's body weight divided by the square of his or her height. In the case of Orientals, when the BMI is 30 or more, it is called extremely obese.

Furthermore, not only weight-related factors but also other factors including the percentage of visceral fat, an abdominal fat percentage and a body fat percentage are measured to eventually result in a diagnosis of extreme obesity.

Such an extremely obese patient may have many health problems. For example, the representative complications of the incidence of diabetes, hyperpiesia, hyperlipidemia, a fatty liver, etc. can rapidly increase in the extremely obese.

Of course, extreme obesity also rapidly increases the mortality rate resulting from such diseases as well as the incidence thereof, compared to simple obesity. In terms of reducing the weight, achieving the aim of weight-loss for the extremely obese is rapidly reduced in probability, in terms of controlling the size of meals with exercise and staying in the weight-lost state for a long period of time, compared to that of simple obesity.

Statistically, extremely obese patients ordinarily try extreme weight-loss programs, but these easily cause the problem of there being a relapse into an extremely obese state.

Such extremely obese patients may be treated by a surgical method. The following cases are among those which are candidates for the surgical treatment: a patient who has experienced failure with typical weight-loss methods, such as alimentotherapy, exercise, and correction of behavior; a patient who has a BMI of 30 or more accompanied by diabetes, hypertension, a high cholesterol disorder, a fatty liver, joint inflammation, a sleep apnea disorder, etc.; a patient who is ultra-extremely obese with a BMI of 35 or more; and a patient who has morbid abdominal obesity which is one of the characteristic obesity styles of Orientals.

Gastrectomy is a representative example of the surgical treatment method. The gastrectomy is a surgical method of turning the stomach into a tube shape in such a way as to reduce a greater curvature portion of the stomach along a lesser curvature portion, while retaining the vestibular region (antrum: the lowermost portion) which governs important digestive functions.

A patient who has undergone such a gastrectomy can easily experience satiety despite eating a small amount of food. Hence, the amount of meals is reduced so that voracious eating can be prevented. The patient who had a morbid and compulsive appetite before the gastrectomy can have normal appetite.

However, after the gastrectomy, the patient must be hospitalized or receive outpatient treatment, and there is the probability of emesis, diarrhea, laparocele, an infection, pneumonia, a respiratory disease, etc. In addition, if inosculation between the stomach and the intestine is defective, digestive fluid may leak out of the stomach.

Furthermore, such problems increase medical expenses because additional expenses resulting from complications may exceed the expenses of the operation and follow-up treatment expenses.

Moreover, when the patient who has received a gastrectomy takes in food, the food comes in contact with the suture region, thus causing pain. If the suture region is contaminated by food, the suture region may become inflamed. If the suture region is reopened by the inflammation or by damage done to the ligation region after the surgery, the patient has to get surgery again.

Meanwhile, in addition to preventing extreme obesity with a gastrectomy, a stent may be used in such a way that food which has passed through the stomach is prevented from mixing with bile or pancreatic juice so that the duodenum cannot absorb the nutrients of the food.

### SUMMARY OF THE INVENTION

Accordingly, the present invention has been made keeping in mind the above problems occurring in the prior art, and an object of the present invention is to provide a stent which is configured such that the stomach of a patient who has undergone a gastrectomy is not brought into direct contact with food, thus preventing the suture region from being contaminated, thereby preventing the suture region from becoming inflamed, and preventing the suture region from being reopened by the inflammation or damage done to the ligation region after the surgery.

Another object of the present invention is to provide a stent which prevents food from coming contact with the suture region so as to avoid the occurrence of an inflammation, thus reducing the time of treatment.

A further object of the present invention is to provide a stent which includes a medical film which is made of PTFE or silicone, thus preventing food from leaking out of the stent, and enhancing the support performance of the stent.

Yet another object of the present invention is to provide a stent which may be implanted before conducting the gastrectomy and is configured such that food that has passed through the stomach is prevented from mixing with bile or pancreatic juice in the duodenum and is directly moved into the small intestine to prevent the duodenum from absorbing the nutrients of the food while the small intestine directly digests the food and absorbs the nutrients, thus minimizing a nutrient absorption rate, thereby preventing the obesity of the patient.

In order to accomplish the above object, the present invention provides a stent, including: a hollow cylindrical stent body formed by weaving at least one first wire made of a super-elastic shape memory alloy such that a plurality of diamond-shaped openings are formed in the cylindrical stent body, the cylindrical stent body comprising an expanded diameter part on a first end thereof, with bent portions formed along circumferences of both ends of the cylindrical stent body; and a support stent having an elastic spherical structure and formed by weaving at least one second wire made of a super-elastic shape memory alloy or by threading the second wire to each other in a zigzag manner such that a hollow hole is formed through a central portion of the elastic spherical structure, the support stent being fitted, through the hollow hole, over a circumferential outer surface of the cylindrical stent body behind the expanded diameter part, with coupling wires connecting both ends of the support stent to the cylindrical stent body. The cylindrical stent body is coated with a first medical film. The expanded diameter part has a rounded portion on a junction between the expanded diameter part and the cylindrical stent body and is coated with a second medical film, with a removal string ring connected to at least one of the bent portions of the expanded diameter part. The support stent is coated with a third medical film. The rounded portion of the expanded diameter part is caught and supported by an inner surface of a connection tube connected between an esophagus and a stomach of a patient, the support stent is supported by a curved inner surface of a cardiac orifice of the stomach, and a second end of the cylindrical stent body is inserted into a pylorus of the patient via a cavity of the stomach.

### BRIEF DESCRIPTION OF THE DRAWINGS

The above and other objects, features and advantages of the present invention will be more clearly understood from the following detailed description taken in conjunction with the accompanying drawings, in which:

FIG. 1 is an exploded perspective view of a stent according to the present invention;

FIG. 2 is a perspective view of the stent according to the present invention;

FIG. 3 is a front view of the stent according to the present invention;

FIG. 4 is a perspective view of the stent provided with a plurality of stent supports according to the present invention;

FIG. 5A is a partial sectional view showing a shape-memory alloy stent wherein a first film is formed inside a first wire made of PTFE according to the present invention;

FIG. 5B is a partial sectional view showing a shape-memory alloy stent wherein the first film is formed outside the first wire made of PTFE according to the present invention;

FIG. 6 is a partial sectional view showing a second or third medical silicone film formed on a wire forming an expanded diameter part or a support stent while closing openings according to the present invention; and

FIG. 7 is a view showing the stent of the present invention implanted in the stomach and the duodenum of a patient after or before gastrectomy is conducted.

### DESCRIPTION OF THE PREFERRED EMBODIMENTS

Hereinafter, a preferred embodiment of the present invention will be described in detail with reference to the attached drawings.

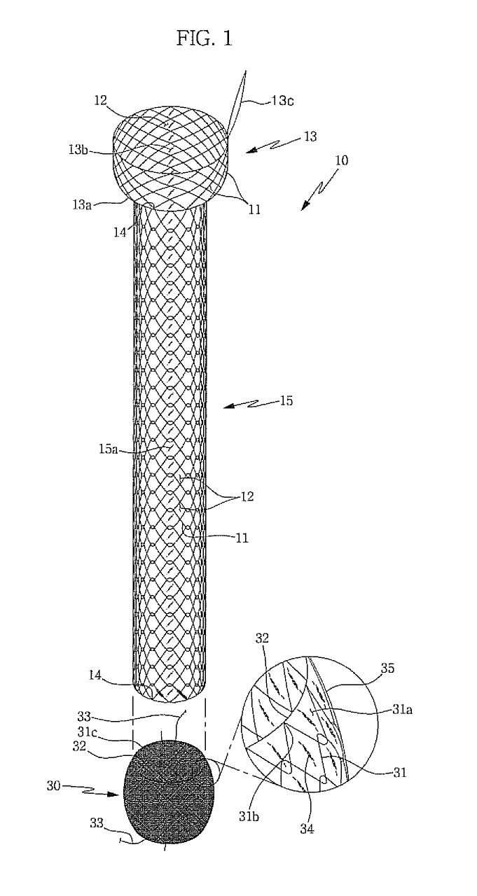

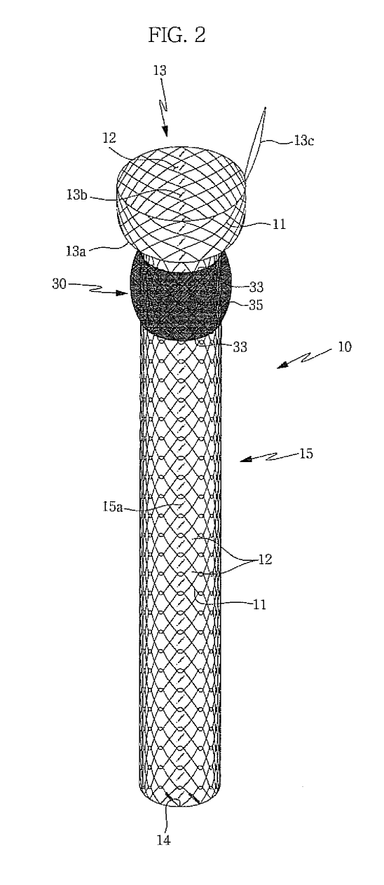

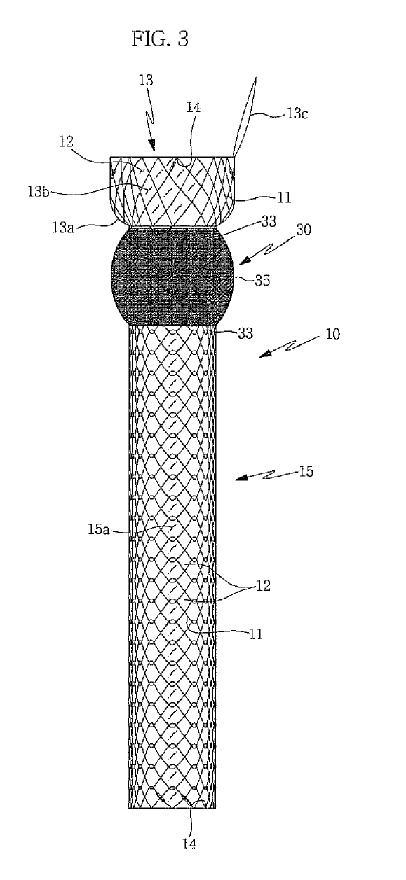

As shown in FIGS. 1 through 3, a stent according to the present invention comprises a shape-memory alloy stent 10 which is formed by weaving a piece of or a plurality of wires 11 made of material such as a super-elastic shape memory alloy such that a plurality of diamond-shaped openings 12 are formed in the stent 10. The shape-memory alloy stent 10 has a hollow cylindrical stent body 15 and an expanded diameter part 13 which is formed on an end of the cylindrical stent body 15. Bent portions 14 are formed along the circumferences of both ends of the cylindrical stent body 15.

The surface of the cylindrical stent body 15 is coated with a first medial film 15a.

Particularly, when the first medial film 15a is formed on the circumferential inner surface of the cylindrical stent body 15, flow friction is reduced which is generated, for example, when contact occurs between the wires 11 and food which has passed through the esophagus 200. When the first medial film 15a is formed on the circumferential outer surface of the cylindrical stent body 15, the wire 11 is prevented from coming into direct contact with the inner surface of a stomach 300.

Furthermore, the expanded diameter part 13 has a rounded portion 13a which is provided on a junction between the expanded diameter part 13 and the cylindrical stent body 15.

The surface of the expanded diameter part 13 is coated with a second medical film 13b.

A removal string ring 13c is connected to the bent portions 14 of the expanded diameter part 13.

The stent of the present invention further includes a support stent 30 which has an elastic spherical structure and is formed by weaving a piece of or a plurality of support stent wires 31 made of material such as a super-elastic shape memory alloy or by threading the support stent wires 31 to each other in a zigzag manner such that a hollow hole 32 is formed through the central portion of the elastic spherical structure.

The support stent 30 is fitted, through the hollow hole 32, over the circumferential outer surface of the cylindrical stent body 15 behind the expanded diameter part 13. Both ends of the support stent 30 are connected to the cylindrical stent body 15 by coupling wires 33. The surface of the support stent 30 is coated with a third medical film 34.

In detail, the support stent 30 comprises a support stent body 31c which is formed by weaving the support stent wire 31 or by threading support stent wire 31 in a zigzag manner such that openings 31a are formed in the support stent body 31c and that bent portions 31b are formed along the circumferences of both ends of the support stent body 31c.

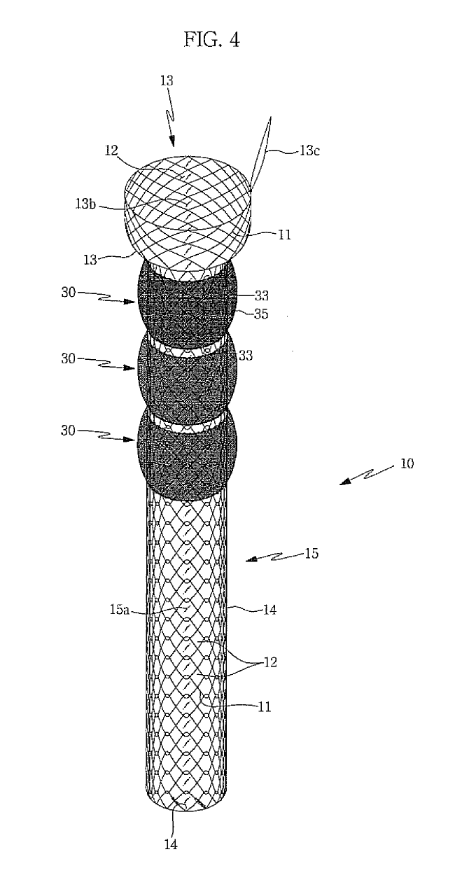

As shown as FIG. 4, a mesh of the support stent 30 formed by weaving the support stent wire 31 is smaller than that of the cylindrical stent body 15 formed by weaving the wires 11, so that the support stent 30 has comparatively superior elasticity. The stent of the present invention may include a plurality of support stents 30.

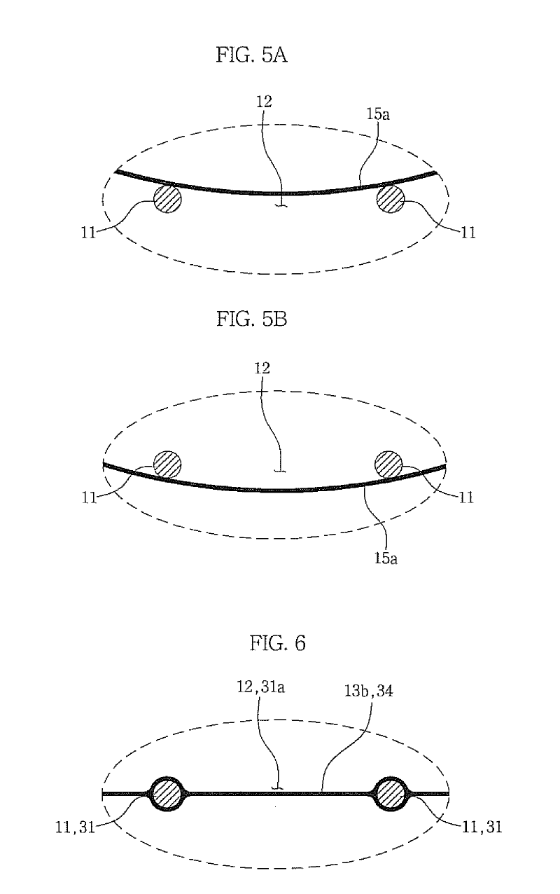

As shown in FIGS. 5A and 5B, the first medical film 15a of the shape-memory alloy stent 10 may be provided on the circumferential inner surface of the wires 11 (refer to FIG. 5A) or, alternatively, it may be provided on the circumferential outer surface of the wires 11 (refer to FIG. 5B).

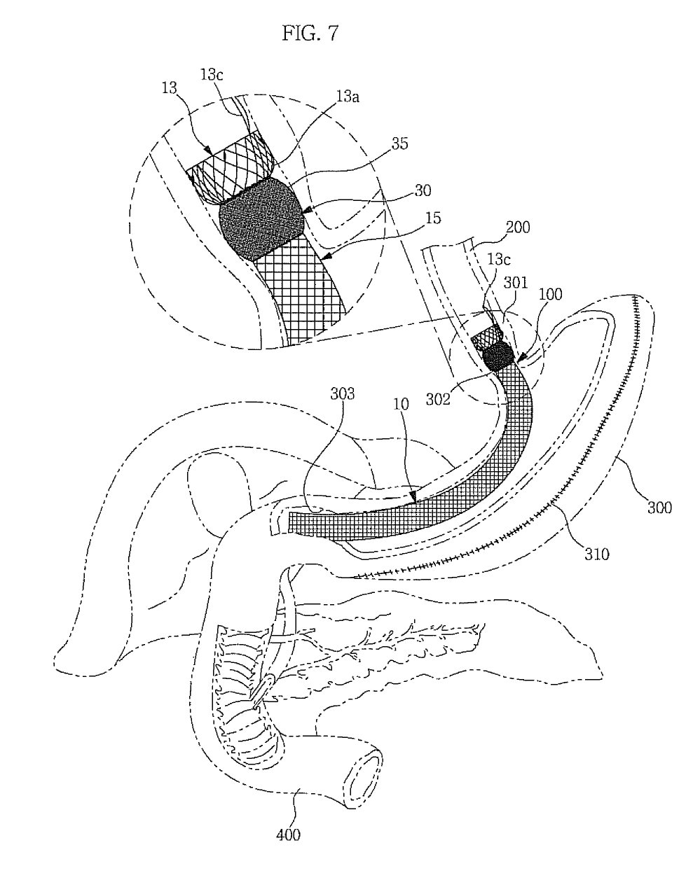

As shown in FIG. 6, the second and third medical films 13b and 34 are respectively formed on the expanded diameter part 13 and the support stent 30 by immersing the expanded diameter part 13 and the support stent 30 in liquefied silicone and hardening them such that the films cover the surfaces of the wires 11 and the support stent wires 31 and the openings defined by the wires 11 and the support stent wires 31.

As shown in FIG. 7, the rounded portion 13a of the expanded diameter part 13 is caught and supported by the inner surface of a connection duct 301 which connects the esophagus 200 to the stomach 300.

In addition, a spherical convex surface 35 of the support stent 30 is supported by a curved inner surface of a cardiac orifice 302 of the stomach 300.

The shape-memory alloy stent 10 is inserted into a pylorus 303 via the cavity of the stomach 300.

The operation of the shape-memory alloy stent 10 of the present invention having the above-mentioned construction will be described.

In an operation of implanting the shape-memory alloy stent 10, a separate stent operation apparatus, such as a catheter, (not shown) is used.

Referring to FIGS. 1 and 7, first, the shape-memory alloy stent 10 is inserted into the catheter with a contracted volume and thereafter is inserted into the esophagus 200 by the catheter.

Then the extended diameter part 13 of the shape-memory alloy stent 10 is caught and supported by the inner surface of the connection duct 301 connecting the esophagus 200 with the stomach 300.

In addition, the spherical convex surface 35 of the support stent 30 is supported by the curved inner surface of the cardiac orifice 302 of the stomach 300.

The lower end of the shape-memory alloy stent 10 that is opposite to the expanded diameter part 13 is inserted into the pylorus 303 via the cavity of the stomach 300.

In this embodiment, the rounded portion 13a of the expanded diameter part 13 is rounded in such a way that the diameter thereof is reduced to the end connected to the shape-memory alloy stent 10 so that the rounded portion 13a is smoothly held and supported by the inner surface of the connection duct 301 that connects the esophagus 200 to the stomach 300.

Thereby, the rounded portion 13a of the expanded diameter part 13 is brought into close contact with the inner surface of the connection duct 301 without any gap being left between them. Therefore, food which has passed through the esophagus 200 is prevented from entering between the expanded diameter part 13 and the inner surface of the cardiac orifice 302.

Moreover, the support stent 30 is also brought into close contact with the inner surface of the cardiac orifice 302 so that the support stent 30 reliably supports the expanded diameter part 13, thus completing the implanting of the shape-memory alloy stent 10.

After the implanting of the shape-memory alloy stent 10 has been completed, food which is supplied into the esophagus 200 moves into the expanded diameter part 13.

Here because the shape-memory alloy stent 10 is configured such that the rounded portion 13a of the expanded diameter part 13 is brought into close contact with the inner surface of the connection duct 301, the food which has passed through the esophagus 200 is prevented from moving into the stomach 300 outside the shape-memory alloy stent 10 due to the weight or the speed at which the food is moving. In other words, all the food moves into the shape-memory alloy stent 10 without leaking between the expanded diameter part 13 and the inner surface of the connection duct 301.

The support stent 30 is closely adhered to the inner surface of the cardiac orifice 302 of the stomach 300 by its own elastic force, preventing the shape-memory alloy stent 10 from becoming displaced from the correct position and from completely moving into the stomach 300.

Meanwhile, because the second medical film 13b of the expanded diameter part 13 and the third medical film 34 of the support stent 30 are made of silicone that are plastic and soft and have an elasticity higher than that of PTFE (polyetrafluoroethylene), the support force of the expanded diameter part 13 and the support stent 30 can be further enhanced.

Food that has moved into the cylindrical stent body 15 via the expanded diameter part 13 directly moves from the shape-memory alloy stent 10 into the duodenum 400.

Food that moves into the duodenum 400 along the shape-memory alloy stent 10 mixes with bile coming out of the gall bladder and pancreatic juice coming out of the pancreas and is digested so that nutrients of the food are absorbed by the body.

Thus, when the patient who has undergone a gastrectomy takes in food, the food moves directly into the duodenum 400 via the stent 100 without meeting the stomach 300, thus preventing inflammation of the excised region of the stomach 300, so that a suture region 310 can be safely protected.

Furthermore, food which has moved into the duodenum 400 via the shape-memory alloy stent 10 is digested so that nutrients of the food are absorbed by the body, thus preventing the nutrient supply from becoming imbalanced.

After the suture region 310 has completely healed after a predetermined period of time has passed, the shape-memory alloy stent 10 is removed from the body. For this, the removal string ring 13c connected to the expanded diameter part 13 is pulled by the catheter. Then, the shape-memory alloy stent 10 is reduced in diameter while increasing in length and is inserted into the catheter before being completely removed from the body of the patient.

As described above, a stent of the present invention prevents the stomach of a patient who has undergone a gastrectomy from being brought into direct contact with food, so that the suture region is prevented from being contaminated, thus preventing inflammation of the suture region, and preventing the suture region from being reopened by the inflammation or damage done to the ligation region after the surgery.

Furthermore, the stent prevents food from coming into contact with the suture region so as to avoid the occurrence of inflammation, thus reducing the treatment period.

In addition, the stent includes a medical film which is made of PTFE or silicone, thus preventing food from leaking out of the stent, and enhancing the support performance of the stent. Moreover, the stent may be implanted before the gastrectomy is conducted. In this case, food that has passed through the stomach is also prevented from mixing with bile or pancreatic juice in the duodenum and directly moves into the small intestine to prevent the duodenum from absorbing nutrients of the food while the small intestine directly digests the food and absorbs the nutrients, thus minimizing a nutrient absorption rate, thereby preventing the obesity of the patient.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A stent, comprising:
a hollow cylindrical stent body formed by weaving at least one first wire made of a super-elastic shape memory alloy such that a plurality of diamond-shaped openings are formed in the cylindrical stent body, the cylindrical stent body comprising an expanded diameter part on a first end thereof, with bent portions formed along circumferences of both ends of the cylindrical stent body; and
a support stent having an elastic spherical structure and formed by weaving at least one second wire made of a super-elastic shape memory alloy or by threading the second wire to each other in a zigzag manner such that a hollow hole is formed through a central portion of the elastic spherical structure, the support stent being fitted, through the hollow hole, over a circumferential outer surface of the cylindrical stent body behind the expanded diameter part, with coupling wires connecting both ends of the support stent to the cylindrical stent body,
wherein the cylindrical stent body is coated with a first medical film,
the expanded diameter part has a rounded portion on a junction between the expanded diameter part and the cylindrical stent body and is coated with a second medical film, with a removal string ring connected to at least one of the bent portions of the expanded diameter part, and
the support stent is coated with a third medical film,
wherein the rounded portion of the expanded diameter part is caught and supported by an inner surface of a connection tube connected between an esophagus and a stomach of a patient, the support stent is supported by a curved inner surface of a cardiac orifice of the stomach, and a second end of the cylindrical stent body is inserted into a pylorus of the patient via a cavity of the stomach.

2. The stent as set forth in claim 1, wherein the first medical film of the hollow cylindrical stent body is made of PTFE (polyetrafluoroethylene) and is formed in a circumferential inner surface or a circumferential outer surface of the first wire.

3. The stent as set forth in claim 1, wherein a mesh of the support stent is smaller than a mesh of the hollow cylindrical stent body, and the support stent comprises a plurality of support stents.

4. The stent as set forth in claim 1, wherein the second and third medical films are respectively formed by immersing the expanded diameter part and the support stent in liquefied silicone and hardening the silicone applied thereto so that the second and third medical films cover surfaces of the first wire of the expanded diameter part and the second wire and close the diamond-shaped openings defined by the first wire of the expanded diameter part and the second wire of the support stent.
